(19) United States
(12) Patent Application Publication
(10) Pub. No.: US 2004/0156814 A1
(43) Pub. Date: Aug. 12, 2004

(54) Cosmetic eye-care compositions comprising a low molecular weight cationic copolymer

(76) Inventors: Christina Ligia Andrianov, Monroe, NY (US); Stewart Todd Elder, Butler, NY (US)

Correspondence Address: CIBA SPECIALTY CHEMICALS CORPORATION, PATENT DEPARTMENT, 540 WHITE PLAINS RD, P O BOX 2005, TARRYTOWN, NY, 10591-9005, US

(21) Appl. No.: 10/771,560

(22) Filed: Feb. 3, 2004

Related U.S. Application Data
(60) Provisional application No. 60/445,738, filed on Feb. 7, 2003.

(51) Int. Cl. A61K 7/06; A61K 7/11

(52) U.S. Cl. 424/70.17

(57) ABSTRACT

Aqueous compositions comprising a cationic copolymer with a molecular weight in the range of 30,000 to 300,000 and a high molecular weight alcohol, for example at least one C₂₀-C₄₀ alcohol, and the use of said aqueous compositions to prepare cosmetic eye-care formulations, for example mascara or eyeliner, are described. The eye-care formulations give outstanding shine as well as durability to rub-off to the cosmetic eye-care compositions.

The present invention relates to cosmetic eye-care compositions such as eyeliners and mascara. More particularly it relates to a cosmetic eye-care composition comprising a low molecular weight cationic copolymer and a high molecular weight alcohol which gives outstanding shine as well as durability to rub-off to eye-care products.

### BACKGROUND OF THE INVENTION

Cosmetic eye-care compositions are pigmented compositions for application to eyelashes and eyelids to beautify the eyes by providing desirable appearance aspects such as thickening, lengthening, coloring, and defining the individual lashes. Compositions applied to the eyelashes are generally referred to as mascara, while compositions applied to the eyebrows are generally referred to as eyeliners.

Mascara and eyeliner compositions in general consist of one or more film formers, pigments and the vehicle (which mostly evaporates when the product is applied to the eye area and allows the film to set).

Mascara is known in a number of forms including cakes or blocks, creams, gels, and low viscosity liquids.

Cake mascaras were originally the most popular form of this cosmetic. They consisted of at least 50% soap, with the pigment mixed into the soap cakes. With a wet brush, the mascara could be lathered and then applied to the lashes, resulting in a satisfactory, smooth application. Its primary drawback was that the film on the lashes was very water soluble and prone to smudging and running, with the product transferring to the skin around the perimeter of the eye. By incorporating waxes to improve their water-resistance, cake mascaras were improved over the original soap-based form. This was usually at the expense of the smoothness of application however.

With the advent of the automatic applicator, spiral-tipped wand or wand and brush applicator, cream and liquid mascaras came into being. Cream mascaras were usually dispersions of waxes and pigments in water with the end consistency very much like a vanishing cream. Combined with an automatic applicator, they soon surpassed the cake mascara in popularity due to their convenience of use, which was less dependent upon actual technique by the user than the cake-based applicators. Most of the ingredients were similar to the improved form of the cake mascara, so many of the same shortcomings were still inherent. However, because it had a creamy texture, the concentration of water was greater. This permitted the incorporation of natural and synthetic film-formers to help improve wear.

Currently there are three general types of mascara formulations on the market:

1) Anhydrous, organic solvent-based suspensions: they are waterproof but not smudge proof. They do not come off with soap and water and are very difficult to remove. They also have a tendency to smudge. These types of formulations have a high oil content. The eye area is also oily, so this type of formulation is not favorable from this perspective.

2) Water-in-Oil emulsions: they are also waterproof but not smudge proof. Some of these can be removed with soap and water.

3) Oil-in-Water emulsions: are “water based” and not waterproof, but they are water resistant. If the film is sufficiently flexible, the mascara can also be “flake proof” and “smudge proof”. Water resistance can be achieved with the addition of emulsion polymers, acrylics, polyvinyl acetates, polyurethanes, etc.

The emulsion types of mascara formulations are essentially free of organic solvents.

The eyeliner compositions currently found on the market are of three types:

1) Pencils: which are the most popular due to their ease of application;

2) Aqueous, hydro-alcoholic suspensions: these are gelled with a cellulose derivative or a natural gum. They are not totally stable. Therefore they require shaking before being used; and

3) Oil-in-water (O/W) emulsions: these are similar to O/W mascaras but in a much thinner, more liquid formulation.

WO-A-97/26860 describes the use of a conventional polyquatemium-7 in a conditioning shampoo formulation. Polyquatemium-7 is a high molecular weight cationic copolymer, more specifically a copolymer of diallyl dimethyl ammonium chloride with acrylamide, for example a 50/50 wt/wt % copolymer, and having a typical molecular weight of more than about 500,000, for example 1 million or more. Typically such polymers are prepared and sold as dilute aqueous solutions, for example about 7-10 weight % solutions.

U.S. Pat. No. 5,925,337 describes the use of a conventional polyquatemium-7 in a solvent-based, emulsifier-free waterproof mascara.

In one aspect the present invention relates to mascara and eyeliner compositions comprising a low molecular weight cationic copolymer and a high molecular weight alcohol. In one embodiment the low molecular weight cationic copolymer is a polyquatemium-7 and the high molecular weight alcohol is lanolin alcohol.

The compositions of the present invention can be fabricated in a multitude of forms, such as creams, pastes and solids. A preferred composition form is an emulsion, for example an oil-in-water emulsion.

The inventive compositions are removable with soap and water and provide excellent shine and wear properties. The inventive composition provides superior performance for mascara formulations, eyeliner formulations and other eye make-up products like cream eyeshadow. More specifically the mascara has more shine, curl, and lash separation and volume deposit than the leading brands of mascara on the market today.

### DETAILED DESCRIPTION OF THE INVENTION

Cationic copolymers with a molecular weight in the range 30,000 to 300,000, in the form of aqueous solutions having a concentration of between 20-50% by weight of active polymer, wherein the solution has a viscosity of less than 10,000 cP (mPa.s) at 25° Celsius, are known from WO 02/40622, the disclosure of which is incorporated by reference in its entirety.

Surprisingly it has now been found that a composition comprising a cationic copolymer with molecular weight in the range 30,000 to 300,000 and a high molecular weight alcohol gives outstanding shine as well as durability to rub-off to cosmetic eye-care compositions such as eyeliner and mascara.

In particular the invention relates the use of said aqueous cationic copolymer solution having a molecular weight in the range 30,000 to 300,000 in conjunction with a high molecular weight alcohol, for example at least one C₂₀-C₄₀ alcohol such as lanolin alcohol, to prepare a cosmetic eye-care formulation, for example a mascara or eyeliner.

The high molecular weight C₂₀-C₄₀ alcohol can be linear, branched or mono- or polycyclic, and can be of natural or synthetic origin. Mixtures containing two or more of these alcohols are preferred. In one embodiment a mixture of naturally occurring alcohols such as lanolin alcohol is employed.

Lanolin alcohol is a complex mixture that comprises three major chemical groups: aliphatic alcohols, sterols (e.g. cholesterol) and trimethyl sterols (e.g. lanosterol and dehydrolanosterol). Lanolin alcohol typically comprises about 26% of aliphatic alcohols, 38% of cholesterol and 27% of trimethyl sterols, the remainder being hydrocarbons and undetermined products. Lanolin alcohol is widely available commercially.

The viscosity of the above aqueous cationic copolymer solutions is measured using a Brookfield RVT viscometer, using spindle 3 at 10 rpm. The molecular weight of the copolymer is determined by conventional gel permeation chromatography (GPC) techniques.

The aqueous copolymer solution preferably has a concentration of 30 to 45% by dry weight. Said aqueous copolymer solution preferably has a viscosity of from 1000 to 10,000 cP at 25° Celsius, measured with a Brookfield RVT viscometer.

The aqueous solution preferably comprises a copolymer which is the reaction product of from 25 to 90% by weight of nonionic monomer units and from 10 to 75% by weight of cationic monomer units, for example from 50 to 80% by weight of nonionic monomer units and 20 to 50% by weight of cationic monomer units and especially from 70 to 80% by weight of nonionic monomer units and 20 to 30% by weight of cationic monomer units.

The nonionic monomer may be any nonionic monomer, with preferred monomers being selected from the group consisting of

unsaturated N-substituted amides, e.g. N-vinyl formamide, N-vinyl caprolactam, and N-vinyl pyrrolidone,

(meth)acrylates having mono- or multi hydroxy functional group(s), for example hydroxylethyl acrylate(HEA), hydroxylethyl methacrylate(HEMA), hydroxypropyl acrylate (HPA), hydroxypropyl methacrylate (HPMA), glycerol mono-acrylate and trimethylolpropane monoacrylate,

acrylamide and its derivatives, for example N-hydroxymethylacrylamide, N-tris(hydroxy- methyl)methylacrylamide, other N-alkyl or N-alkoxy substituted acrylamides, for example N,N-dimethyl acrylamide and acrylamide derivatives.

A preferred nonionic monomer is acrylamide or a derivative thereof.

The cationic monomer may be any cationic monomer, with preferred cationic monomers being selected from the group consisting of dialkylaminoalkyl (meth)acrylamide and preferably, acrylate, as acid addition or, preferably, quaternary ammonium salts, and diallyl dialkyl ammonium halides.

Preferred acrylates and methacrylates are di-C₁-C₄aminoethyl (meth)acrylates. Preferred acrylamides and (meth)acrylamides are di-C₁-C₄alkyl aminoalkyl (meth)acrylamides, in particular dimethylaminoethyl (meth)acrylate (DMAE(M)A) and dimethylaminopropyl (meth)acrylamide (DMAP(M)A), with the respective methacrylate and methacrylamide compounds being particularly preferred, as acid addition and, preferably quaternary ammonium salts.

A particularly preferred cationic monomer is diallyl dimethyl ammonium chloride (DADMAC).

A particularly preferred copolymer comprises the reaction product of from 70 to 80% by weight of acrylamide nonionic monomer units and 20 to 30% by weight of diallyl dimethyl ammonium chloride cationic monomer units.

The copolymer has a molecular weight from about 30,000 to 300,000, preferably about 40,000 to 250,000. An especially preferred molecular weight range is from 100,000 to 200,000, for example about 150,000.

The aqueous copolymer solution may have a pH of any value, however a preferred pH range is from pH 3 to pH 7.5. The most preferred pH values are from pH 4.5 to pH 6.5.

The cationic content of the copolymer, based on 100% active polymer, may range from 0.6 to 4.5 moles/kg.

The aqueous copolymer solution may comprise some unreacted monomer resulting from the copolymerization of the nonionic and cationic monomers, for example less than 20 ppm.

The aqueous copolymer solution may further comprise a preservative, such as those commonly used in cosmetics, drugs and foods. Such preservatives include phenoxyethanol, benzoic acid, esters of p-hydroxybenzoic acid, DMDM hydantoin, imidazolidinyl urea, diazodinyl urea, methylchloroisothiazolinone, methylisothiazolinone. Preferred preservatives include phenoxyethanol and p-hydroxybenzoic acid propyl ester and salts thereof and p-hydroxybenzoic acid methyl ester and salts thereof.

Aqueous solutions of a cationic copolymer with a molecular weight in the range of 30,000 to 300,000, having a concentration of 20 to 50% by weight and a viscosity of less than 10,000 cP at 25° Celsius, may be produced by known polymerization processes, for example by a continuous addition polymerization technique as taught in WO 02/40622.

In one embodiment the mixture of a cationic copolymer with a molecular weight in the range of 30,000 to 300,000 and a high molecular weight alcohol is present in an emulsion.

An emulsion is considered to be a heterogeneous system, consisting of at least one immiscible liquid dispersed in another in the form of droplets. It is a composition consisting of at least two distinct phases known as the internal phase and the external phase. The material(s) of the “internal phase” are dispersed as small droplets or solid particles within another distinct phase of the emulsion composition. The term “external phase” refers to the continuous phase in which the internal phase is dispersed. The term “emulsion” as used herein includes dispersions, wherein the internal phase comprises solid particles at normal use temperatures.

The cosmetic eye-care compositions according to the present invention will normally contain, in addition to a volatile liquid vehicle, which is essentially water, additional ingredients that are known in the art to be useful in cosmetics such as eyeliner and mascara. Said ingredients include various materials which are nonvolatile, at least at ambient temperatures. Many are solid or semi solid in their normal state and at ambient temperatures. Said materials include, but are not necessarily limited to water-soluble, film-forming polymers other than those described above, waxes, fats, oils, pigments, dyes, gums, resins, inorganic and organic materials, fillers, thickeners, gelling agents, and mixtures thereof, which are conventionally incorporated into eye makeup compositions.

The nonvolatile components, which include the low molecular weight cationic copolymer and high molecular weight alcohol according to the invention, comprise from about 30% to about 60%, for example from about 35% to about 55%, and especially from about 40% to about 50% of the present composition. The volatile liquid vehicle, which is essentially water, comprises from about 30% to about 65%, for example from about 40% to about 55% of the composition of the present invention.

The water-soluble, film-forming polymers comprise polymers selected from the group consisting of cellulose derivatives, and synthetic polymers made by various polymerization techniques. Preferred cellulose derivatives are selected from the group consisting of hydroxyethylcellulose, hydroxypropylcellulose, hydroxypropryl methylcellulose and mixtures thereof. Specific water-soluble, film-forming polymers useful in the present invention include, but are not necessarily limited to Natrosol 250HHR (hydroxyethylcellulose) from Aqualon/-Hercules. Water-soluble synthetic polymers include polymers such as polyvinylpyrrolidone (PVP), for example PVP K-30 from ISP, quaternized polyvinylpyrrolidones and high molecular weight cationic copolymers, for example a conventional copolymer of diallyl dimethyl ammonium chloride with acrylamide (polyquatemium-7) having a typical molecular weight of more than about 500,000, for example 1 million or more. Examples of the latter include Salcare® SC10 and Salcare® SC 11 of Ciba Specialty Chemicals.

The water-soluble, film-forming polymers comprise from about 2% to about 25% by weight, for example from about 3% to about 15% by weight and especially from about 4% to about 10% by weight of the composition of the present invention, with the proviso that from about 10% to 100% of the film-forming polymer comprises the low molecular weight cationic copolymer according to the invention.

The high molecular weight alcohol comprises from about 0.1% to about 10% by weight, for example from about 0.2% to about 5% by weight and especially from about 0.3% to about 3% by weight of the composition of the present invention.

The cosmetic eye care compositions of the present invention include oil-in-water emulsion compositions. These compositions require lipophilic materials that form the internal phase of the composition. These lipophilic materials usually are waxes and fats generally known for use in the cosmetic preparations.

Fats that are normally used can be derived from animal, vegetable and synthetic sources, and mixtures thereof. Fats have a melting point anywhere from 55° C. to about 100° C. and a needle penetration as measured according to the American standard ASTM D5, from about 3 to about 40 at 25° C. Preferably the fats selected for use in the present invention are fatty acid esters, which are solids at room temperature and exhibit crystalline structure. Examples of fatty acid esters useful in the present invention include the glyceryl esters of higher fatty acids such as stearic, palmitic, glyceryl monostearate, glyceryl distearate, glyceryl tribehenate and mixtures thereof. When present, they are typically used at levels from about 0.1% to about 10% by weight, for example from about 0.2% to about 5% and especially from about 0.3% to about 3% by weight of the composition of the present invention.

Waxes are defined as lower-melting organic mixtures or compounds of high molecular weight, solid at room temperature and generally similar in composition to fats and oils except that they contain no glycerides. Some are hydrocarbons, others are esters of fatty acids and alcohols. Waxes useful in the present invention are selected from the group consisting of animal waxes, vegetable waxes, mineral waxes, synthetic waxes, petroleum waxes, ethylenic polymers, hydrocarbon types such as Fischer-Tropsch waxes, silicone waxes, and mixtures thereof wherein the waxes have a melting point between 55° and 100° C. and a needle penetration, as measured according to the American standard ASTM D5, of 3 to 40 at 25° C.

The specific waxes useful in the present invention are selected from the group consisting of beeswax, lanolin wax, shellac wax (animal waxes); camauba, candelilla, bayberry (vegetable waxes); ozokerite, ceresin, (mineral waxes); paraffin, microcrystalline waxes (petroleum waxes); polyethylene, (ethylenic polymers); polyethylene homopolymers (Fischer-Tropsch waxes); C₂₄-C₄₅ alkyl methicones (silicone waxes); and mixtures thereof. Preferred waxes include beeswax, camauba wax and mixtures thereof.

The waxes are typically used at levels from about 5% to about 30% by weight, for example from about 6% to about 20% and in particular from about 8% to about 15% by weight in the composition of the present invention.

The solids component of the cosmetic eye care compositions of the present invention usually contains cosmetically acceptable pigments selected from the group consisting of inorganic pigments, organic lake pigments, pearlescent pigments, and mixtures thereof. When employed, the pigments are present in proportions that depend upon the color and the intensity of the color which it is intended to produce.

The level of pigments in the composition of present invention is from about 3% to about 20%, for example from about 5% to about 15% and especially from about 6% to about 12% by weight. Said pigments may optionally be surface-treated by methods known in the art, such as treatments with silicones, perfluorinated compounds, lecithin, and amino acids.

Inorganic pigments useful in the present invention include those selected from the group consisting of rutile or anatase titanium dioxide, coded in the Color Index under the reference CI 77,891; black, yellow, red and brown iron oxides, coded under references CI 77,499, 77, 492 and, 77,491; manganese violet (CI 77,742); ultramarine blue (CI 77,007); chromium oxide (CI 77,288); chromium hydrate (CI 77,289); and ferric blue (CI 77,510) and mixtures thereof. Black iron oxide is particularly preferred.

The pearlescent pigments useful in the present invention include those selected from the group consisting of the white pearlescent pigments such as mica coated with titanium oxide, bismuth oxychloride, colored pearlescent pigments such as titanium mica with iron oxides, titanium mica with ferric blue, chromium oxide and the like, titanium mica with an organic pigment of the above-mentioned type as well as those based on bismuth oxychloride and mixtures thereof.

Emulsifiers may also be used in the present invention to assist in the preparation of homogenous compositions and to improve their stability. Said emulsifiers include, but, are not necessarily limited to the group consisting of soaps, phosphate esters, ethoxylated alcohols, ethoxylated fatty acids, ethoxylated fatty esters, polyol ether esters, glycerol esters, sucrose or sorbitan esters, glucose esters, potassium or DEA-cetyl phosphate, fatty esters and mixtures thereof. Alkanolamine salts of fatty acids, for example triethanolamine stearate, are preferred.

The emulsifiers are typically used at levels from about 1% to about 10% by weight, for example from about 2% to about 8% by weight in the composition of the present invention.

An optional component of the cosmetic eye care compositions of the present invention is one or more preservatives to protect against the growth of potentially harmful microorganisms. Preservatives especially useful in the present compositions include imidazolidinyl urea, diazolidinyl urea, quaternium-15, methylparaben, ethylparaben, propylparaben, butylparaben, EDTA, quatemary ammonium compounds, phenoxyethanol and mixtures thereof. These preservatives are defined in detail in the CTFA Cosmetic Ingredient Dictionary, Third Edition, which is incorporated herein by reference. Of these preservatives, phenoxyethanol, methylparaben, propylparaben, and mixtures thereof are particularly preferred for use in the inventive composition. The preferred concentration of preservatives used in the present compositions is in the range of between about 0.01% and about 2% by weight, for example between about 0.1% and about 1.2% by weight based on the total weight of the composition.

Still another optionally included component in the composition of this invention is a sequestering agent. A sequestering agent is preferably present in a concentration in the range of between about 0.01% and about 0.1% by weight, based on the total weight of the composition. The preferred sequestering agents for use in this composition include disodium EDTA and trisodium EDTA. These agents are completely identified in the aforementioned CTFA Cosmetic Ingredient Dictionary, Third Edition. Of these, trisodium EDTA is particularly preferred for use in the composition.

The cosmetic eye care compositions of the present invention can be prepared by conventional mixing techniques that are well known in the cosmetics industry. The following examples illustrate some of these techniques.

These examples describe certain embodiments of this invention, but the invention is not limited thereto. It should be understood that numerous changes to the disclosed embodiments can be made in accordance with the disclosure herein without departing from the spirit or scope of the invention. These examples are therefore not meant to limit the scope of the invention. Rather, the scope of the invention is to be determined only by the appended claims and their equivalents. In these examples all parts given are by weight unless otherwise indicated. All parts are by weight on an as is basis unless otherwise indicated.

### EXAMPLE 1

Preparation of mascara emulsions.

| Phase | INCI Name | Trade Name | Supplier | A | B | C |
|---|---|---|---|---|---|---|
| A | Water (Aqua) | Deionized Water | N/A | 54.60 | 54.60 | 54.60 |
| | Polyvinylpyrolidone | PVP K-30 | ISP | 1.80 | 1.80 | 1.80 |
| | Hydroxyelthylcellulose | Natrosol 250HHR/CS | Hercules | 0.30 | 0.30 | 0.30 |
| | Triethanolamine 99% | TEA | Dow | 2.00 | 2.00 | 2.00 |
| | Methylparaben | Nipagin M | Clariant | 0.20 | 0.20 | 0.20 |
| | Black Iron Oxide | C33-5198 | Sun Chem. | 10.00 | 10.00 | 10.00 |
| B | Stearic Acid | Emersol 132 NF | Cognis | 4.50 | 4.50 | 4.50 |
| | Glyceryl Monostearate | Cerasynt GMS | ISP | 2.00 | 2.00 | 2.00 |
| | White Beeswax | Bleached Beeswax Prills | Ross | 7.00 | 7.00 | 7.00 |
| | Copernicia Cerifera | Yellow Canauba Wax | Ross | 4.00 | 4.00 | 4.00 |
| | (Camauba) Wax | Flakes | | | | |
| | Lanolin alcohol | Jeelan Alcohol | JEEN | 1.00 | 1.00 | 0.00 |
| | Hydroxylated Lanolin | Ritahydroxy | RITA | 0.00 | 0.00 | 1.00 |
| | Propylparaben | Nipasol M | Clariant | 0.10 | 0.10 | 0.10 |
| C | Polyquaternium-7 | Salcare ® Super 7* | Ciba | 12.00 | 0.00 | 12.00 |
| | Polyquaternium-7 | Salcare ® SC 10* | Ciba | 0.00 | 12.00 | 0.00 |
| D | Phenoxyethanol | Phenoxyethanol | Clariant | 0.50 | 0.50 | 0.50 |
| | | | Total | 100.00 | 100.00 | 100.00 |

*Ciba Salcare ® Super 7 is a polyquaternium-7 having a molecular weight of 100,000-200,000;
*Ciba Salcare ® SC 10 is a polyquaternium-7 having a molecular weight of about 1,500,000.

Procedure:

The water of phase A is weighed into a mixing vessel. While stirring with a homogenizer, the hydroxyelthylcellulose and PVP are sprinkled into the water. The mixture is heated to 80-85° C. and homogenization is maintained for one hour. Then the remaining ingredients of phase A are added to the aqueous mixture. Upon addition of the pigment the batch is homogenized for an additional hour.

The ingredients of phase B are added into a separate vessel and heated to 85° C; then mixed well with a propeller until a clear, yellow solution is obtained. Phase B is then slowly added to phase A while maintaining homogenization and the temperature as mentioned above.

The resulting oil-in-water emulsion is then cooled. At 65° C. stirring is switched to side sweep mixing using a blade stirrer and continued with moderate agitation. At this point phase C is added. At 55-60° C. phase D is added and, as the mixture thickens up, agitation is slowed. The emulsion is mixed until it reaches room temperature.

Results:

Each of the three mascara formulations were evaluated on synthetic and human eyelashes and compared with a current market leader. The results are described below:

| Sample | Observations |
|---|---|
| A | Sample A was very shiny and had a deep |
| | black appearance. In addition, sample A |
| | provided the most shine of all 4 mascara |
| | samples when applied to the eyelashes. |
| | The eyelashes were separated and well |
| | defined. Volume and curl of the lashes |
| | was better than the market leader. |
| B | Sample B was not as shiny as A, and |
| | had a grayish-black color. When |
| | sample B was applied to the eyelashes |
| | they appeared clumpy and slightly sticky. |
| C | Sample C (no lanolin alcohol) had |
| | an appearance similar to sample B. |
| | It was also clumpy when applied to |
| | the eyelashes. |

### EXAMPLE 2

Preparation of an eyeliner. Black Eyeliner formulation

| Seq. | INCI Name | Trade Name | Supplier | Percent |
|---|---|---|---|---|
| A | DI Water | N/A | N/A | 49.55 |
| | Dimethicone | DC 2501 | Dow | 0.05 |
| | Copolyol | Cosmetic Wax | Coming | |
| | Polyvinyl- | PVP K-30 | ISP | 2.00 |
| | pyrrolidone | | | |
| | Black Iron | C33-5198 | Sun | 10.00 |
| | Oxide | | Chemical | |
| | Hydroxyethyl- | Natrosol 250HR CS | Hercules | 0.30 |
| | cellulose | | | |
| | Disodium EDTA | EDETA-BD | BASF | 0.05 |
| B | Triethanolamine | TEA | DOW | 0.80 |
| | 99%, USP | | | |
| | DI Water | N/A | N/A | 3.00 |
| C | 1,3-Butylene Glycol | Jeechem BUGL | JEEN | 4.00 |
| | Methylparaben | Nipagin M | Clariant | 0.20 |
| D | Beeswax | White Bleached | Ross | 5.00 |
| | | Beeswax Prills | | |
| | Copemicia | Yellow Camauba Wax | Ross | 4.00 |
| | Cerifera | Flakes | | |
| | Stearic Acid | Emersol -132 NF | Cognis | 1.50 |
| | Isostearic Acid | Liponate ISA Special | Lipo | 0.50 |
| | Cetyl Alcohol | Crodacol C-95 NF | Croda | 0.75 |
| | Lanolin Alcohol | Jeelan Alcohol | Jeen | 1.00 |
| | Propylparaben | Nipasol M | Clariant | 0.10 |
| E | Polyquaternium -7 | Salcare° Super 7 | CIBA | 15.00 |
| F | Diazolidinyl Urea | Germal II | ISP | 0.20 |
| | DI Water | N/A | N/A | 2.00 |

| | |
|---|---|
| Total | 100.00 |

Procedure:

Phase D is heated in a separate vessel to 85-90° C. and stirred until a clear solution is obtained. The water of phase A is weighed into a mixing vessel. While stirring with a homogenizer, the remaining ingredients of Phase A are added in order while heating to 85-87° C. The batch is then homogenized for 1 hour. Phase B is added to A; then phase C to A. Phase D is then added to A while homogenization and temperature is maintained for 15 minutes. The resulting oil-in-water emulsion is cooled down to about 55° C.; then phase E is added using a paddle mixer with moderate agitation. At 45° C. Phase F is added to the batch. The batch is cooled until it reaches 30° C.

### EXAMPLE 3

Preparation of mascara emulsions.

| Phase | INCI Name | Trade Name | Supplier | A | B |
|---|---|---|---|---|---|
| A | Water (Aqua) | Deionized Water | N/A | 61.10 | 38.60 |
| | Polyvinylpyrolidone | PVP K-30 | ISP | 1.80 | 1.80 |
| | Hydroxyelthylcellulose | Natrosol 250HHR/CS | Hercules | 0.30 | 0.30 |
| | Triethanolamine 99% | TEA | Dow | 2.00 | 2.00 |
| | Methylparaben | Nipagin M | Clariant | 0.20 | 0.20 |
| | Black Iron Oxide | C33-5198 | Sun Chem. | 8.00 | 8.00 |
| B | Stearic Acid | Emersol 132 NF | Cognis | 4.50 | 4.50 |
| | Glyceryl Monostearate | Cerasynt GMS | ISP | 2.00 | 2.00 |
| | White Beeswax | Bleached Beeswax Prills | Ross | 7.00 | 7.00 |
| | Copemicia Cerifera | Yellow Canauba Wax Flakes | Ross | 4.00 | 4.00 |
| | (Camauba) Wax | | | | |
| | Lanolin alcohol | Jeelan Alcohol | JEEN | 1.00 | 1.00 |
| | Hydroxylated Lanolin | Ritahydroxy | RITA | 0.00 | 0.00 |
| | Propylparaben | Nipasol M | Clariant | 0.10 | 0.10 |
| C | Polyquatemium-7 (3%) | Salcare ® Super 7*(40% Active) | Ciba | 7.50 | 0.00 |
| | Polyquatemium-7 (3%) | Salcare ® SC 10* (10% Active) | Ciba | 0.00 | 30.00 |
| D | Phenoxyethanol | Phenoxyethanol | Clariant | 0.50 | 0.50 |
| | | | Total | 100.00 | 100.00 |

*Ciba Salcare ® Super 7 is a polyquaternium-7 having a molecular weight of 100,000-200,000;
*Ciba Salcare ® SC 10 is a polyquaternium-7 having a molecular weight of about 1,500,000.

Procedure:

The emulsions are prepared by the procedure of Example 1.

Test Results:

Each of the two mascara formulations was evaluated on synthetic and human eyelashes and compared with a current market leader. The results are described below:

| Sample | Observations |
|---|---|
| A | Sample A was very shiny and had a deep |
| | black appearance. In addition, |
| | sample A provided the most shine of |
| | all the mascara samples evaluated when |
| | applied to the eyelashes. The eyelashes |
| | were separated and well defined. Volume |
| | and curl of the lashes was better than |
| | the market leader. |
| B | Sample B was just as shiny as sample A, |
| | but it was very stringy. When applied |
| | to the lashes it left clumps due to the |
| | “thread-like” strings that formed on |
| | application. Also less volume is |
| | observed on the lashes in comparison |
| | to sample A. |
| Market leader | Samples A and B were compared to one |
| | of the top market leading mascaras. |
| | Sample A is outstanding in volume, |
| | shine and curl in comparison to the |
| | market leading mascara and to sample B. |

What is claimed is:

1. A composition comprising a cationic copolymer with molecular weight in the range of 30,000 to 300,000 and a high molecular weight alcohol.

2. A composition according to claim 1, wherein the high molecular weight alcohol is at least one C₂₀-C₄₀ alcohol.

3. A composition according to claim 2, wherein the at least one high molecular weight C₂₀-C₄₀ alcohol is linear, branched or mono- or polycyclic, or a mixture thereof.

4. A composition according to claim 1, wherein the high molecular weight alcohol is lanolin alcohol.

5. A composition according to claim 1, wherein the cationic copolymer comprises a copolymer which is the reaction product of from 25 to 90% by weight of nonionic monomer units and from 10 to 75% by weight of cationic monomer units.

6. A composition according to claim 5, wherein the nonionic monomer units are selected from the group consisting of unsaturated N-substituted amides, (meth)acrylates having mono- or multi hydroxy functional group(s), and acrylamide and its derivatives.

7. A composition according to claim 5, wherein the cationic monomer units are selected from the group consisting of diallyl dialkyl ammonium halides, dialkyl aminoalkyl (meth)acrylamides, dialkyl aminoalkyl (meth)acrylates, and acid addition and quatemary ammonium salts thereof.

8. A composition according to claim 5, wherein the copolymer comprises the reaction product of from 70 to 80% by weight of acrylamide nonionic monomer units and 20 to 30% by weight of diallyl dimethyl ammonium chloride cationic monomer units.

9. A composition according to claim 5, wherein the copolymer has a molecular weight of from about 40,000 to 250,000.

10. A composition according to claim 1, wherein the mixture of the cationic copolymer and the high molecular weight alcohol is present in an emulsion.

11. A composition according to claim 10, which is an oil-in-water emulsion.

12. A composition according to claim 10, which is a cosmetic eye-care composition.

13. A composition according to claim 12, which is a mascara or eyeliner.

14. A cosmetic eye-care composition according to claim 12, which comprises additional ingredients selected from the group consisting of water-soluble, film-forming polymers other than cationic copolymers with a molecular weight in the range of 30,000 to 300,000, waxes, fats, oils, pigments, dyes, gums, resins, inorganic and organic materials, fillers, thickeners, gelling agents, and mixtures thereof.

15. A composition according to claim 14, in which the water-soluble, film-forming polymers comprise from about 2% to about 25% by weight of the composition, with the proviso that from about 10% to 100% by weight of the film-forming polymers is a cationic copolymer with a molecular weight in the range of 30,000 to 300,000.

16. A composition according to claim 14, in which the water-soluble, film-forming polymers other than cationic copolymers with a molecular weight in the range of 30,000 to 300,000 are selected from the group consisting of cellulose derivatives and synthetic polymers.

17. A composition according to claim 16, in which the cellulose derivatives are selected from the group consisting of hydroxyethylcellulose, hydroxypropylcellulose, hydroxypropryl methylcellulose and mixtures thereof.

18. A composition according to claim 16, in which the synthetic polymers are selected from the group consisting of polyvinylpyrrolidone, quaternized polyvinylpyrrolidone and high molecular weight cationic copolymers.

19. A composition according to claim 16, in which the synthetic polymer is a copolymer of diallyl dimethyl ammonium chloride with acrylamide.

20. A cosmetic eye-care composition according to claim 14, which is an oil-in-water emulsion comprising by weight:
from about 2% to about 25% of a water-soluble, film-forming polymer, with the proviso that
from about 10% to 100% of the film-forming polymer is a cationic copolymer with a molecular weight in the range of 30,000 to 300,000;
from about 0.1% to about 10% of at least one C₂₀-C₄₀ alcohol;
from about 5% to about 30% of a wax;
from about 0.1% to about 10% of a fat, and
from about 3% to about 20% of at least one pigment.

21. A method of improving the properties of a cosmetic eye-care composition, which comprises incorporating a composition according to claim 1 therein.
